(19) United States
(12) Patent Application Publication
(10) Pub. No.: US 2012/0010159 A1
(43) Pub. Date: Jan. 12, 2012

(54) COMBINATION THERAPIES WITH COX-2 INHIBITORS AND TREPROSTINIL

(75) Inventors: Martine Rothblatt, Satellite Beach, FL (US); Martin Auster, Silver Spring, MD (US)

(73) Assignee: United Therapeutics Corporation

(21) Appl. No.: 13/176,085

(22) Filed: Jul. 5, 2011

Related U.S. Application Data
(60) Provisional application No. 61/362,987, filed on Jul. 9, 2010.

(51) Int. Cl. A61K 38/06; A61K 31/415; A61K 31/42; A61K 31/196; A61P 19/02; A61K 38/05; A61K 31/661; A61K 38/07; A61P 29/00; A61K 31/365; A61K 31/216

(52) U.S. Cl. 514/21.9; 514/473; 514/406; 514/378; 514/567; 514/510; 514/21.91; 514/114

(57) ABSTRACT

The present invention is directed to compositions and methods for pain management, and for treating inflammation or an inflammation-associated disorder in a subject comprising administering to the subject a therapeutically effective amount of a COX-2 inhibitor and a therapeutically effective amount of a prostacyclin analog, such as treprostinil, a pharmaceutically acceptable salt thereof, or a treprostinil derivative described herein.

### CROSS REFERENCE TO RELATED PATENT APPLICATIONS

The present application claims the benefit of U.S. provisional application No. 61/362,987, filed Jul. 9, 2010, which is incorporated herein by reference in its entirety.

### BACKGROUND OF THE INVENTION

Non-steroidal anti-inflammatory drugs, usually abbreviated to NSAIDs, are drugs with analgesic, antipyretic and anti-inflammatory effects. As analgesics, NSAIDs are unusual in that they are non-narcotic. NSAIDs are sometimes also referred to as non-steroidal anti-inflammatory agents/analgesics (NSAIAs) or non-steroidal anti-inflammatory medicines (NSAIMs).

NSAIDs can be broadly classified based on their chemical structure. NSAIDs within a group will tend to have similar characteristics and tolerability. There is little difference in clinical efficacy between the NSAIDs when used at equivalent doses. Rather, differences between compounds tended to be with regards to dosing regimens (related to the compound's elimination half-life), route of administration, and tolerability profile.

The most prominent members of this group of drugs are aspirin and ibuprofen. Aspirin type compounds (“salicylates”) include: Aspirin, Amoxiprin, Benorilate, Choline magnesium salicylate, Diflunisal, Faislamine, Methyl salicylate, Magnesium Salicylate and Salicyl salicylate (salsalate). Arylalkanoic acids include: Diclofenac, Aceclofenac, Acemetacin, Bromfenac, Etodolac, Indometacin, Nabumetone, Sulindac and Tolmetin, and 2-Arylpropionic acids (profens) include: Ibuprofen, Carprofen, Fenbufen, Fenoprofen, Flurbiprofen, Ketoprofen, Ketorolac, Loxoprofen, Naproxen, Tiaprofenic acid and Suprofen.

These “traditional” NSAIDs, however, have been known to cause significant gastrointestinal irritation, which can be serious enough to cause gastric injury, including serious ulcers and gastrointestinal bleeding. Certain precautions can be taken to reduce the chances of gastric injury, such as by advising patients to take NSAIDs only after consuming a meal and/or drinking water, and by limiting the dose of the NSAID and duration over which it is administered.

Drugs that are specifically designed to inhibit COX-2 are a newer class of NSAIDs but have fewer gastrotoxic effects. COX-2 inhibitors inhibit the conversion of arachidonic acid to cyclic endoperoxidases PGG₂ and PGH₂, and subsequent isomerization to prostacyclin by the action of prostacyclin synthase. Like other NSAIDS, COX-2 inhibitors decrease inflammation, pain and swelling.

Recently, however, certain adverse cardiovascular events were noted with COX-2 inhibitors, thereby off-setting the benefit of reduced gastrointestinal effects of these selective inhibitors. Current explanations for the increased cardiovascular associated morbidity and mortality are not conclusive.

Thus, there is still a need in the art for effective pain management compositions and improved treatments of inflammatory disease states and disorders that do not have the adverse side effects associated with prior art compounds. The invention is directed to these, as well as other, important ends.

### SUMMARY OF THE INVENTION

The inventors of the present invention surprisingly discovered that administering a combination of a COX-2 inhibitor and a prostacyclin analog or co-administering a COX-2 inhibitor and a prostacyclin analog, such as treprostinil and beraprost, provides a safer, more effective alternative to COX-2 inhibitors alone and other NSAIDS in a pain management regimen and in the treatment of inflammation and an inflammation associated disease or disorder. By administering the combination of these two drugs or co-administering them, the adverse cardiovascular events caused by a COX-2 inhibitor alone can be minimized, since the prostacyclin analog helps to offset the inhibition of endogenous prostacyclin production brought about by administration of the COX-2 inhibitor. Co-administration of prostacyclin analog such as treprostinil or beraprost with a COX-2 inhibitor creates a pain management strategy with less cardiovascular event risk than COX-2 inhibitors alone while maintaining other advantages COX-2 inhibitors possess over other NSAIDs.

Thus, in one embodiment of the present invention is directed to a method of treating inflammation or an inflammation-associated disorder in a subject comprising administering to a subject in need thereof a therapeutically effective amount of a cyclooxygenase-2 (COX-2) inhibitor and a therapeutically effective amount of a prostacyclin analog.

In another embodiment, the present invention is directed to a method of managing pain in a subject comprising administering to a subject in need thereof a therapeutically effective amount of a cyclooxygenase-2 (COX-2) inhibitor and a therapeutically effective amount of a prostacyclin analog represented by the following structural formula:

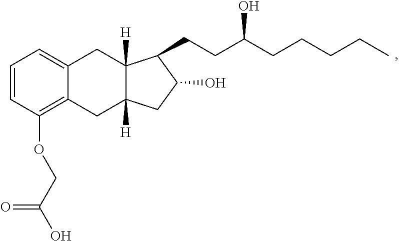

or a pharmaceutically acceptable salt or a derivative thereof.

In yet another embodiment, the present invention is directed to a composition comprising a therapeutically effective amount of a COX-2 inhibitor and a therapeutically effective amount of a prostacyclin analog.

### DETAILED DESCRIPTION OF THE EMBODIMENTS

### Definitions

Unless otherwise specified, “a” or “an” means “one or more.”

As used herein, the terms “subject” and “patient” are used interchangeably and refer to a mammal, preferably a human.

As used herein, a “pharmaceutically acceptable salt” refers to a salt that is useful in preparing a pharmaceutical composition and is generally safe, non-toxic and neither biologically nor otherwise undesirable pharmaceutical use.

As used herein, the terms “therapeutically effective dose” or “therapeutically effective amount” means a dose that produces the desired effect for which it is administered. The exact dose will be ascertainable by one skilled in the art using known techniques, and efficacy can be measured in conventional ways. For example, for pain management, efficacy can, for example, be measured by assessing the time to reduce, minimize or alleviate the pain, or time to onset of next pain episode. For arthritis-induced pain management, efficacy can additionally be measured by assessing the freedom of motion. The therapeutically effective dose may be adjusted depending on conditions of disease, the age, body weight, general health conditions, sex, and diet of the subject, dose intervals, administration routes, excretion rate, and combinations of drugs. Exemplary therapeutically effective doses for treprostinil, pharmaceutically acceptable salts, and treprostinil derivatives include, but are not limited to, 0.125 mg BID to 16 mg BID. Exemplary therapeutically effective doses for COX-2 inhibitor include, but are not limited to, about 0.01 to about 100 mg/kg body weight per day, preferably about 0.1 to about 50 mg/kg per day, more preferably about 1 to 20 mg/kg per day. Exemplary therapeutically effective doses for COX-2 inhibitor also include, but are not limited to, about 50 mg to about 200 mg twice a day, such as 50 mg, 100 mg or 200 mg twice a day.

The terms “treating”, “treatment”, and the like are used herein to refer to obtaining a desired therapeutic or prophylactic effect. The effect is therapeutic in terms of a partial or complete cure of a condition or symptom related to inflammation or inflammation-associated disorders (e.g., pain), or adverse effect attributed to, for example, a disease or condition. The term “treatment”, as used herein, covers any treatment in a mammal, particularly a human, and includes: (a) preventing a condition or symptom related to inflammation or inflammation-associated disorders (e.g., pain) from recurring in a subject or (b) alleviating inflammation or inflammation-associated disorders (e.g., pain), e.g., reducing the progression of inflammation or inflammation-associated disorders (e.g., pain). The compositions and methods disclosed herein can be used for treatment of inflammation or inflammation-associated disorders (e.g., pain) and/or prophylactic management of inflammation or inflammation-associated disorders (e.g., pain).

Prostacyclin analogs are compounds that have similar biological activities as prostacyclin, such as promoting the production cyclic AMP (cAMP). Prostacyclin analogs include known prostacyclin analogs in the art, such as iloprost, cisaprost, beraprost and trerprostinil, or a combination of such analogs. In one embodiment, the prostacyclin analogs are long-duration prostacyclin analogs, such as iloprost, beraprost and trerprostinil. In a preferred embodiment, the prostacyclin analog is beraprost, treprostinil, a pharmaceutically acceptable salt or derivative thereof. In another preferred embodiment, the prostacyclin analog is treprostinil, a pharmaceutically acceptable salt or derivative described herein.

Co-administration of the COX-2 inhibitor with a prostacyclin analog (combination therapy) encompasses administering a single pharmaceutical composition comprising the two drugs together or administering two or more separate pharmaceutical compositions, one comprising the COX-2 inhibitor and the other(s) comprising the prostacyclin analog. Further, although co-administration or combination therapy preferably means that the two therapeutic agents are administered at the same time as one another, it also encompasses instances in which the two therapeutic agents are administered at different times but in such a way that their therapeutic effects overlap.

An “inflammation-associated disorder” is a disease or a condition that is associated with inflammation. Exemplary inflammation-associated disorders include, but are not limited to, pain, headaches, fever, arthritis (including but not limited to rheumatoid arthritis, spondyloarthropathies, gouty arthritis, osteoarthritis, systemic lupus erythematosus and juvenile arthritis), asthma, bronchitis, menstrual cramps, tendinitis, bursitis, and skin related conditions such as psoriasis, eczema, burns and dermatitis.

Generally, reference to a certain element such as hydrogen or H is meant to include all isotopes of that element. For example, if an R group is defined to include hydrogen or H, it also includes deuterium and tritium.

The phrases “oral bioavailability” and “bioavailability upon oral administration” as used herein refer to the systemic availability (i.e., blood/plasma levels) of a given amount of drug administered orally to a patient.

The phrase “unsubstituted alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms. More preferred such unsubstituted alkyl groups have from 1 to 10 carbon atoms while even more preferred such groups have from 1 to 5 carbon atoms. Most preferred unsubstituted alkyl groups include straight and branched chain alkyl groups having from 1 to 3 carbon atoms and include methyl, ethyl, propyl, and —CH(CH₃)₂.

The phrase “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; and oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles. Preferred substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine, diarylamine, heterocyclylamine, (alkyl)(heterocyclyl)amine, (aryl)(heterocyclyl)amine, or diheterocyclylamine group.

The phrase “unsubstituted arylalkyl” refers to unsubstituted alkyl groups as defined above in which a hydrogen or carbon bond of the unsubstituted alkyl group is replaced with a bond to an aryl group as defined above. For example, methyl (—CH₃) is an unsubstituted alkyl group. If a hydrogen atom of the methyl group is replaced by a bond to a phenyl group, such as if the carbon of the methyl were bonded to a carbon of benzene, then the compound is an unsubstituted arylalkyl group (i.e., a benzyl group). Thus the phrase includes, but is not limited to, groups such as benzyl, diphenylmethyl, and 1-phenylethyl (—CH(C₆H₅)(CH₃)) among others.

The phrase “substituted arylalkyl” has the same meaning with respect to unsubstituted arylalkyl groups that substituted aryl groups had with respect to unsubstituted aryl groups. However, a substituted arylalkyl group also includes groups in which a carbon or hydrogen bond of the alkyl part of the group is replaced by a bond to a non-carbon or a non-hydrogen atom. Examples of substituted arylalkyl groups include, but are not limited to, —CH₂C(═O)(C₆H₅), and —CH₂(2-methylphenyl) among others.

The present invention is directed to combination therapies for treating inflammation or an inflammation-associated disorder in a subject, or use of the combination in a pain management regimen. The combination therapy comprises administering to the subject, either concurrently or sequentially, a therapeutically effective amount of a COX-2 inhibitor and a therapeutically effective amount of a prostacyclin analog, such as treprostinil, a pharmaceutically acceptable salt thereof, or a derivative of treprostinil.

The combination therapies of the present invention also would be useful to treat gastrointestinal conditions such as inflammatory bowel disease, colorectal polyps, Crohn's disease, gastritis, irritable bowel syndrome and ulcerative colitis and for the prevention of colorectal cancer. In addition, the combination therapies of the present invention would be useful in treating inflammation in such diseases as vascular diseases, migraine headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, gingivitis, hypersensitivity, conjunctivitis, swelling occurring after injury, myocardial ischemia, and the like.

In one embodiment, the combination therapies of the present invention are for use in treating inflammation and inflammation-associated disorders. In one embodiment, the inflammation-associated disorder is arthritis. In another embodiment, the inflammation-associated disorder is pain. In yet another embodiment, the inflammation-associated disorder is fever.

A “COX-2” inhibitor refers to a molecule that inhibits the activity of the enzyme cyclooxygenase 2, an enzyme responsible for inflammation and pain. Preferred COX-2 inhibitors include those that are selective for COX-2 (e.g., have a 10 fold higher affinity for COX-2 compared to COX-1), which include, but are not limited to, rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6-dimethyl-6H-dibenzo[b,d]pyran carboxylic acid (CT-3), 2(5H)-furanone, 5,5-dimethyl-(1-methylethoxy)-[4-(methylsulfonyl)phenyl]-(DFP); carprofen (RIMADYLO), (acetyloxy)-benzoic acid, 3-[(nitrooxy)methyl]phenyl ester (NCX4016), P54 (CAS Reg. No. 1309960) 2,6-bis(1,1-dimethylethyl) [(E)-(2-ethyl-1,1-dioxoisothiazolidinylidene)methyl]phenol (S-2474), 5(R)-thio sulfonamide-3(2H)-benzofuranone (SVT-2016) and N-[3-(formyl-amino)oxo-phenoxy-4H benzopyranyl]methanesulfonamide (“T-614”), or a pharmaceutically acceptable salt thereof. In one embodiment, the COX-2 inhibitor is selected from the group consisting of rofecoxib, celecoxib, valdecoxib, and lumiracoxib. In another embodiment, the COX-2 inhibitor is rofecoxib. Alternatively, the COX-2 inhibitor is celecoxib.

Compounds (such as treprostinil) with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts), alkaline earth metal salts (such as magnesium and calcium salts) and salts formed with amine containing compounds such as trimethylamine, triethylamine, pyridine, picoline, ethanolamine, diethanolamine, triethanolamine, choline, glucosamine, ethylenediamine, lysine, arginine and ornithine. Compounds with basic groups, such as amine groups, can form pharmaceutically acceptable salts with pharmaceutically acceptable acid(s). Suitable pharmaceutically acceptable acid addition salts include salts of inorganic acids (such as hydrochloric acid, hydrobromic, hydroboric acid, phosphoric, metaphosphoric, nitric and sulfuric acids) and of organic acids (such as, formic acid, acetic acid, trifluoroacetic acid, fumaric acid, aspartic acid, glutamic acid, benzensulfonic, benzoic, citric, ethanesulfonic, fumaric, gluconic, glycolic, isethionic, lactic, lactobionic, maleic, malic, methanesulfonic, succinic, p-toluenesulfonic, oxalic, lactic, citric, and tartaric acids).

In one embodiment, for combination therapies of the present invention, the prostacyclin analog is treprostinil or a pharmaceutically acceptable salt thereof. Preferably, the pharmaceutically acceptable salt of treprostinil is a sodium salt or a diethanolamine salt.

In another embodiment, for combination therapies of the present invention, the prostacyclin analog is a treprostinil derivative or a pharmaceutically acceptable salt thereof. A “treprostinil derivative” is a compound that is similar to treprostinil in structure and biological activity. In one embodiment, the treprostinil derivative is a treprostinil derivative.

In one embodiment, treprostinil derivatives are those described in U.S. Pat. No. 7,544,713, the entire disclosure of which is incorporated herein by reference. These treprostinil derivatives are represented by structural formula (I):

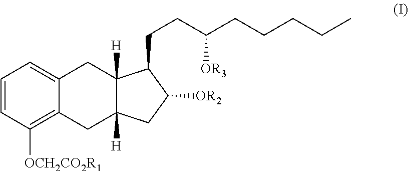

or a pharmaceutically acceptable salt thereof, wherein,
R¹ is independently selected from the group consisting of H, substituted and unsubstituted alkyl groups, arylalkyl groups and groups wherein OR¹ form a substituted or unsubstituted glycolamide ester;
R² and R³ may be the same or different and are independently selected from the group consisting of H, phosphate and groups wherein OR² and OR³ form esters of amino acids or proteins, provided that R¹, R² and R³ are not all H;
an enantiomer of the compound; and
a pharmaceutically acceptable salt of the compound.

When OR¹ form a substituted or unsubstituted glycolamide ester, R¹ can be —CH₂CONR⁴R⁵, wherein R⁴ and R⁵ may be the same or different and are independently selected from the group consisting of H, OH, substituted and unsubstituted alkyl groups, —(CH₂)ₘCH₃, —CH₂OH, and —CH₂(CH₂)ₙOH, wherein m is 0, 1, 2, 3 or 4, and n is 0, 1, 2, 3 or 4.

R¹ can be a C₁-C₄ alkyl group, such as methyl, ethyl, propyl or butyl. Alternatively, R¹ is a substituted or unsubstituted benzyl groups, such as —CH₂C₆H₅, —CH₂C₆H₄NO₂, —CH₂C₆H₄OCH₃, —CH₂C₆H₄Cl, —CH₂C₆H₄(NO₂)₂, or —CH₂C₆H₄F. The benzyl group can be ortho, meta, para, ortho/para substituted and combinations thereof. Suitable substituents on the aromatic ring include halogens (fluorine, chlorine, bromine, iodine), —NO₂ groups, —OR¹⁶ groups wherein R¹⁶ is H or a C₁-C₄ alkyl group, and combinations thereof.

Alternatively, when R¹ is —CH₂CONR⁴R⁵ then R⁴ and R⁵ may be the same or different and are independently selected from the group consisting of H, OH, —CH₃, and —CH₂CH₂OH. When R¹ is not H, generally one or both of R² and R³ are H.

In one embodiment, one or both of R² and R³ are H and R¹ is —CH₃, —CH₂C₆H₅. In another embodiment, when one or both of R² and R³ are H, then R¹ is —CH₂CONR⁴R⁵, and one or both of R⁴ and R⁵ are H, —OH, —CH₃, —CH₂CH₂OH.

In one embodiment, when one or both of R² and R³ are not H, R² and R³ can be independently selected from phosphate and groups wherein OR² and OR³ are esters of amino acids, dipeptides, esters of tripeptides and esters of tetrapeptides. In some embodiments, only one of R² or R³ is a phosphate group. In some embodiments, where at least one of R² and R³ is not H, generally R¹ is H. In other embodiments, one of R² or R³ is H and the other R² or R³ is as defined elsewhere herein. In some embodiments, R² is H and R³ is not H. In additional embodiments, R¹ and R³ are H and R² is a group wherein OR² is an ester of an amino acid or a dipeptide. In further embodiments, R¹ and R² are H and R³ is a group wherein OR³ is an ester of an amino acid or a dipeptide.

In certain embodiments, where one or both of the OR² and OR³ groups form esters of amino acids or peptides, i.e., dipeptides, tripeptides or tetrapeptides, these can be depicted generically as —COCHR⁶NR⁷R⁸ wherein R⁶ is selected from the group consisting of amino acid side chains, R⁷ and R⁸ may be the same or different and are independently selected from the group consisting of H, and —COCHR⁹NR¹⁰R¹¹. In the embodiments wherein the amino acid is proline, R⁷ together with R⁶ forms a pyrrolidine ring structure. R⁶ can be any of the naturally occurring amino acid side chains, for example —CH₃ (alanine), —(CH₂)₃NHCNH₂NH (arginine), —CH₂CONH₂ (asparagine), —CH₂COOH (aspartic acid), —CH₂SH (cysteine), —(CH₂)₂CONH₂ (glutamine), —(CH₂)₂COOH (glutamic acid), —H (glycine), —CHCH₃CH₂CH₃ (isoleucine), —CH₂CH(CH₃)₂ (leucine), —(CH₂)₄NH₂ (lysine), —(CH₂)₂SCH₃ (methionine), —CH₂Ph (phenylalanine), —CH₂OH (serine), —CHOHCH₃ (threonine), —CH(CH₃)₂ (valine),

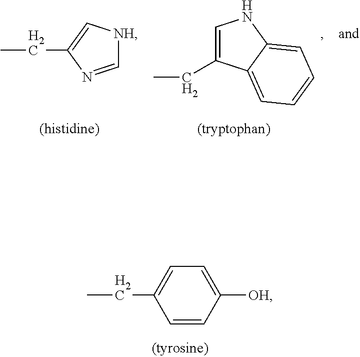

—(CH₂)₃NHCONH₂ (citrulline) or —(CH₂)₃NH₂ (ornithine). Ph designates a phenyl group.

In the above embodiments, R⁷ and R⁸ may be the same or different and are selected from the group consisting of H, and —COCHR⁹NR¹⁰R¹¹, wherein R⁹ is a side chain of amino acid, R¹⁰ and R¹¹ may be the same or different and are selected from the group consisting of H, and —COCHR¹²NR¹³R¹⁴, wherein R¹² is an amino acid side chain, R¹³ and R¹⁴ may be the same or different and are independently selected from the group consisting of H, and —COCHR¹⁵NH₂. One skilled in the art will realize that the peptide chains can be extended on the following scheme to the desired length and include the desired amino acid residues.

In the embodiments where either or both of OR² and OR³ groups form an ester of a peptide, such as dipeptide, tripeptide, tetrapeptide, etc. the peptides can be either homopeptides, i.e., repeats of the same amino residue, or heteropeptides, i.e., made up of different combinations of amino acids.

As will be understood by the skilled artisan when only one of R⁷ and R⁸ includes a peptide bond to further amino acid, such as in the di, tri and tetrapeptides, the resulting peptide chain will be linear. When both R⁷ and R⁸ include a peptide bond, then the peptide can be branched.

In still other embodiments, R¹ is H and one of R² or R³ is a phosphate group or H while the other R² or R³ is a group such the OR² or OR³ is an ester of an amino acid, such as an ester of glycine or alanine

Generally, the compounds of structural formula (I) described herein have enhanced oral bioavailability compared to the oral bioavailability of treprostinil, either in free acid or salt form. The described compounds can have oral bioavailability that is at least 25%, 50% 100%, 200%, 400% or more compared to the oral bioavailability of treprostinil. The absolute oral bioavailability of these compounds can range between 10%, 15%, 20%, 25%, 30% and 40%, 45%, 50%, 55%, 60% or more when administered orally. For comparison, the absolute oral bioavailability of treprostinil is on the order of 10%, although treprostinil sodium has an absolute bioavailability approximating 100% when administered by subcutaneous infusion.

The COX-2 inhibitor for use in the combination therapies of the present invention is administered either sequentially or concurrently with a therapeutically effective amount of a prostacyclin analog, such as treprostinil, a pharmaceutically acceptable salt thereof or a treprostinil derivative described herein.

For concurrent administration, the COX-2 inhibitor and prostacyclin analog can be administered at the same time in different formulations, or prepared together as a single formulation. For example, a pharmaceutical composition including one or more active agents is formulated in accordance with routine procedures as a pharmaceutical composition adapted for intravenous, subcutaneous, nasal, intramuscular or other administration to human beings described herein. In one embodiment, an oral formulation is prepared.

The COX-2 inhibitor and the prostacyclin analog, such as treprostinil, a pharmaceutically acceptable salt thereof or a treprostinil derivative described herein, can be administered by any route by which the compound will be bioavailable in therapeutically effective amounts including oral and parenteral routes. The compounds can be administered intravenously, topically, subcutaneously, intranasally, rectally, intramuscularly, transdermally or by other parenteral routes. When administered orally, the compounds can be administered in any convenient dosage form including, for example, capsule, tablet, liquid, suspension, and the like. In one embodiment, treprostinil, a pharmaceutically acceptable salt thereof, or a treprostinil derivative described herein can be administered using a metered inhaler, as described in US 2008/0200449, the entire teaching of which is incorporated herein by reference.

The pharmaceutical compositions of the invention can also include a pharmaceutically acceptable carrier. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

Pharmaceutical compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

Tests have shown that treprostinil, and in particular the compounds of structure (I) have increased bioavailability when delivered to the duodenum. Accordingly, one embodiment of the present invention involves preferential delivery of the desired compound to the duodenum as well as pharmaceutical formulations that achieve duodenal delivery. Duodenal administration can be achieved by any means known in the art. In one of these embodiments, the present compounds can be formulated in an enteric-coated dosage form. Generally, enteric-coated dosage forms are usually coated with a polymer that is not soluble at low pH, but dissolves quickly when exposed to pH conditions of 3 or above. This delivery form takes advantage of the difference in pH between the stomach, which is about 1 to 2, and the duodenum, where the pH tends to be greater than 4.

The formulations of the invention may be designed for to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations may be compressed into pellets or cylinders and implanted intramuscularly or subcutaneously as depot injections or as implants such as stents. Such implants may employ known inert materials such as silicones and biodegradable polymers.

The present invention, thus generally described, will be understood more readily by reference to the following examples, which are provided by way of illustration and are not intended to be limiting of the present invention.

### EXAMPLES

### Example 1

### Decreased Adverse Effects for Combination Treatment with COX-2 Inhibitor and Treprostinil

In a pre-clinical study, cells treated with COX-2 inhibitor express lower levels of prostacyclin synthase and accordingly show a reduction of stimulation of the prostacyclin receptor. See, e.g., Kapoor et al., PNAS (1999), 96 (1): 272-277. Others have shown that COX-2 inhibitors inhibit prostacyclin synthase activity. Griffoni et al., J. Cell. Mol. Med. 2007, 11 (2): 327-338; Schildknecht et al., FASEB J. 2004, 18 (6): 757-759. Concomitant administration of a prostacyclin analog with a COX-2 inhibitor is therefore expected to preserve appropriate prostacyclin receptor activation and normal function.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method of treating inflammation or an inflammation-associated disorder in a subject comprising co-administering to a subject in need thereof a therapeutically effective amount of a cyclooxygenase-2 (COX-2) inhibitor and a therapeutically effective amount of a prostacyclin analog.

2. The method of claim 1, wherein the prostacyclin analog is beraprost, treprostinil or a pharmaceutically acceptable salt or derivative thereof.

3. The method of claim 2, wherein the prostacyclin analog is represented by the following structural formula: 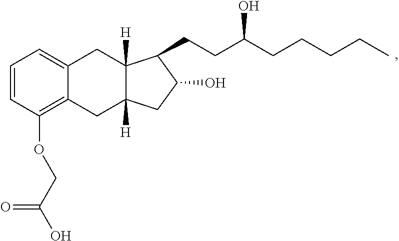
or a pharmaceutically acceptable salt or a derivative thereof.

4. The method of claim 3, wherein the prostacyclin analog is treprostinil or a pharmaceutically acceptable salt thereof.

5. The method of claim 4, wherein the prostacyclin analog is a sodium salt or a diethanolamine salt of treprostinil.

6. The method of claim 3, wherein the prostacyclin analog is represented by the following structural formula: 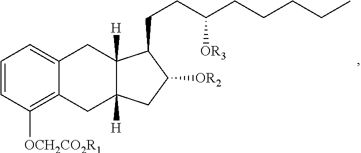
or a pharmaceutically acceptable salt thereof, wherein:
R¹ is independently selected from the group consisting of H, substituted and unsubstituted alkyl groups, arylalkyl groups, and groups wherein OR¹ are substituted or unsubstituted glycolamide esters; and
R² and R³ may be the same or different and are independently selected from the group consisting of H, phosphate and groups wherein OR² and OR³ form esters of amino acids or proteins, provided that R¹, R² and R³ are not all —H.

7. The method of claim 6, wherein when OR¹ forms a substituted or unsubstituted glycolamide ester, R¹ is —CH₂CONR⁴R⁵, wherein R⁴ and R⁵ may be the same or different and are independently selected from the group consisting of H, OH, substituted and unsubstituted alkyl groups, —(CH₂)ₘCH₃, —CH₂OH, and —CH₂(CH₂)ₙOH, wherein m is 0, 1, 2, 3 or 4, and n is 0, 1, 2, 3 or 4.

8. The method of claim 7, wherein R¹ is a C₁-C₄ alkyl group.

9. The method of claim 8, wherein R¹ is selected from the group consisting of methyl, ethyl, propyl or butyl.

10. The method of claim 6, wherein R¹ is a substituted or unsubstituted benzyl group.

11. The method of claim 10, wherein R¹ is CH₂C₆H₅.

12. The method of claim 7, wherein one or both of R⁴ and R⁵ are independently selected from the group consisting of H, —OH, —CH₃, or —CH₂CH₂OH.

13. The method of claim 6, wherein one or both of R² and R³ are H.

14. The method of claim 6, wherein one or both of R² and R³ are not H and R² and R³ are independently selected from phosphate and groups wherein OR² and OR³ are esters of amino acids, dipeptides, esters of tripeptides and esters of tetrapeptides.

15. The method of claim 6, wherein only one of R² or R³ is a phosphate group.

16. The method of claim 14, wherein R² and R³ are independently selected from groups wherein OR² and OR³ are esters of amino acids.

17. The method of claim 16, wherein one or both of R² and R³ are esters of glycine or alanine.

18. The method of claim 14 wherein R¹ is H.

19. The method of claim 14, wherein one of R¹ and R² is H.

20. The method of claim 19, wherein R² is H.

21. The method of claim 1, wherein the COX-2 inhibitor is selected from the group consisting of rofecoxib, celecoxib, valdecoxib, and lumiracoxib.

22. The method of claim 21, wherein the COX-2 inhibitor is refecoxib or celecoxib.

23. The method of claim 1, wherein the method is for use in treatment of inflammation.

24. The method of claim 1, wherein the method is for use in treatment of an inflammation-associated disorder.

25. The method of claim 24, wherein the inflammation-associated disorder is arthritis.

26. The method of claim 24, wherein the inflammation-associated disorder is pain.

27. The method of claim 24, wherein the inflammation-associated disorder is fever.

28. A method for managing pain in a subject comprising administering to a subject in need thereof a therapeutically effective amount of a cyclooxygenase-2 (COX-2) inhibitor and a therapeutically effective amount of a prostacyclin analog represented by the following structural formula: 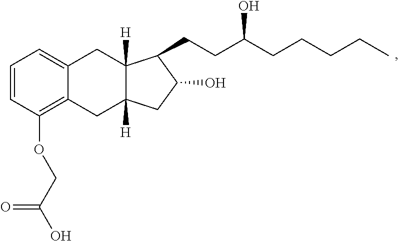
or a pharmaceutically acceptable salt or a derivative thereof.

29. A composition comprising a therapeutically effective amount of a COX-2 inhibitor and a therapeutically effective amount of a prostacyclin analog.

30. The composition of claim 29, wherein the prostacyclin analog is treprostinil, a derivative of treprostinil, or a pharmaceutically acceptable salt of treprostinil.
